(19) United States
(12) Patent Application Publication
(10) Pub. No.: US 2006/0173473 A1
(43) Pub. Date: Aug. 3, 2006

(54) Endoscope comprising a longitudinally guided everting tube

(76) Inventor: Konstantin Bob, Weinheim (DE)

Correspondence Address: MAYER, BROWN, ROWE & MAW LLP, P.O. BOX 2828, CHICAGO, IL, 60690-2828, US

(21) Appl. No.: 11/342,456

(22) Filed: Jan. 30, 2006

(30) Foreign Application Priority Data
Feb. 1, 2005 (DE) 10 2005 004 622.3

(51) Int. Cl. A61B 17/08

(52) U.S. Cl. 606/153

(57) ABSTRACT

The present invention deals with an endoscope comprising an everting tube including a radially outer portion and a radially inner portion. The everting tube surrounds at least partly an endoscope shaft. Longitudinal guiding means which are engaged with each other are formed at each of the endoscope shaft and the everting tube. One of the longitudinal guiding means can be designed such that it forms an undercut, whereas the other is correspondingly shaped so that both longitudinal guiding means are in sliding engagement with each other. Moreover, the longitudinal guiding means can be, on the one hand, in the form of a continuous conveyor disposed at the endoscope shaft and, on the other hand, in the form of an engaging means disposed at the everting tube, which are engaged with each other.

### BACKGROUND OF THE INVENTION

1. Field of the Invention

The present invention relates to a design of an endoscope comprising an everting tube and an endoscope shaft each being provided with a longitudinal guiding means. The present invention especially relates to an endoscope comprising a guided everting tube which is made to slidingly engage with the endoscope shaft by the fact that one of the longitudinal guiding means forms an undercut which is in sliding mesh with the correspondingly shaped other longitudinal guiding means. Furthermore the present invention relates to an endoscope in which the longitudinal guiding means are designed as a continuous conveyor at the endoscope shaft and as an engaging means at the everting tube.

2. Discussion of the Prior Art

Endoscopes are substantially used for visually examining the esophagus, the stomach, the intestines from the stomach or from the anus, the urethra as well as the bladder. For this purpose, the endoscope may be equipped at its distal end with a lighting means and with an optical means, preferably a camera chip, which is connected through lines inside the endoscope shaft to a camera control at the proximal end of the endoscope shaft. The camera control, in turn, is connected via a video processor to an external monitor on which the physician in charge can recognize the areas to be examined. The distal end of the endoscope shaft to be inserted in the hollow canal is formed to be bendable in all directions and can be manually bent similarly to a finger by means of a handle, preferably via two control wheels including a brake at the rear end portion of the endoscope. Moreover, as a rule at least two passages opening at the foremost tip of the distal end extend through the endoscope shaft. On demand, through these passages on the one hand cleansing fluid for cleaning a location to be examined or CO₂ (air) for expanding the hollow canal can be pressed or for changing various operating instruments, for instance forceps or scissors for taking tissue specimens, biopsy needles, heatable cutting wires, coagulating electrodes, can be pushed via a working passage, which instruments can be manually operated at the proximal end of the endoscope shaft likewise via operating wires or Bowden wires inside the internal passage. In the case of taking a tissue specimen, after the distal end has reached the respective location, for instance forceps are inserted at the proximal end of the endoscope in the passage and pushed forward to the distal end. After having taken the specimen, the forceps are withdrawn and removed from the passage so that the further examination can be continued.

In general, the endoscope has an elongated tube-like shape with a diameter of approx. 9 to 15 mm and consists of a bendable material in order to be capable of tracking the curvatures of the hollow canal to be examined, for instance intestinal loops. However, the conventional endoscope bulges, despite an elongated tube-like shape, the intestines to a certain extent in the case of major curvatures of the same so as to adapt to the course of the intestines during the inserting movement and the progressive movement of the endoscope into the hollow canal. This can be very painful to the patient to be examined.

Therefore, an endoscope known from prior art was developed which makes use of an everting tube for an inner shaft. This everting tube can be inverted at both ends thereof or else at one end only, whereby the everting tube can be divided into a radially outer portion, an everting portion and a radially inner portion. The radially outer portion of the everting tube is in contact with a wall of the hollow canal, for instance the intestinal wall, or is adjacent to the same. Thus, the radially outer portion of the everting tube is in a rest position or in a non-moved state relative to the endoscope shaft, as will be described hereinafter. The radially inner portion of the everting tube is in contact with a shaft (endoscope shaft) inserted in the everting tube, wherein upon movement of the endoscope shaft the radially inner portion is made to move while the radially outer portion is in the rest position.

In order to move the endoscope shaft and the everting tube, respectively, in the hollow canal, an everting tube drive, as it is called, is known inter alia.

In this type of drive, at the everting tube a drive means is provided which moves the radially inner portion of the everting tube into an inserting direction, while the radially outer portion of the everting tube is in rest position. Since the endoscope shaft is in contact with the radially inner portion of the everting tube, it is moved along with the everting tube. Thus the endoscope shaft is made to move by the everting tube, the everting tube adapting to the curvatures of the hollow canal, for instance the intestines, and conforms the same by a quasi “rolling motion”. In this way the intestines are prevented from bulging due to the quasi “unrolling motion”. Since the radially outer portion of the everting tube is adjacent to the wall of the hollow canal, for instance the intestinal wall, and thus is in a rest position and the radially inner portion of the everting tube is moved along by its quasi “unrolling motion”, the endoscope shaft necessarily moves more quickly in accordance with the laws of kinematics than the everting portion of the everting tube can progress. In order to compensate this difference in speed, in the solution according to prior art a front stop and a rear stop, between which the everting tube is arranged, are disposed at the endoscope shaft. In this way the endoscope shaft can be prevented from leading ahead of the everting tube and from bulging the intestinal wall as in the case of a common endoscope.

However, the solution according to prior art is based on the problem that due to the use of such stops an increased friction occurs between the latter and the everting tube, whereby the drive power of the drive means is deteriorated. Furthermore, it is not possible by the afore-described arrangement to move the endoscope shaft independently and relative to the everting tube, respectively.

Moreover, from prior art, for example according to DE-199 20 717 A1, an everting tube design of the afore-mentioned species is known. This known design includes a tube inverted both at the distal and the proximal end portions of an endoscope, the tube preferably consisting of a silicone material and coating an endoscope shaft at least in its central portion. Preferably also a drive means is provided which drives the everting tube in the longitudinal direction of the endoscope. To this end, the drive means has a housing to which the opposed free ends of the radially outer portion of the everting tube are fixed and from which force transmitting means, for instance in the form of drive wheels, tracks or shoes, protrude which exert a drive force on the radially inner portion of the everting tube and thus partly also on the endoscope shaft provided in the same.

The everting tube transmits this drive force partly to the endoscope shaft by the fact that the distal/proximal end portion of the everting tube is axially supported in a sliding manner on a radial projection provided at the endoscope shaft and thus forces the endoscope shaft forward upon a bulging movement. In order to prevent the everting tube from forming scales or faults caused by the resulting forces, the known everting tube includes an additional reinforcement, for instance in the form of a laminated coil spring or a tissue. It is also known to pressure-fill the cavity formed by the everting tube and the drive housing with a fluid, for instance an oil, and in this way to reduce a loss of friction and at the same time to inflate the everting tube.

All these measures entail the problem, however, that they reduce the flexibility of the entire endoscope and thereby increase the bending radius thereof which can be minimally obtained.

Finally it is known already from prior art to force-guide an everting tube portion at its distal everting portion. The endoscope shaft equipped with this everting tube includes at its distal end portion a radially circumferential undercut which is superimposed by the distal everting portion of the everting tube. Inside the everting tube in the area of the distal everting portion a snap ring is positioned which is arranged in the mounted state of the endoscope behind the undercut of the endoscope shaft and thus prevents the distal everting portion from withdrawing. The snap ring is supported with play inside the distal everting portion so as to permit a slip-through of the everting tube upon a propelling motion of the shaft. In addition, in the distal end portion of the endoscope shaft a permanent magnet which exerts an external magnetic attraction on the snap ring and thus extends the everting tube in the longitudinal direction is optionally arranged.

Although by the above-described configuration the everting tube can be prevented from forming scales, such a solution has the problem of a high friction between the snap ring and the everting tube, however, whereby the drive force to be applied to the everting tube increases.

Therefore it is an object of the present invention to provide a design of an endoscope which is adapted to solve the afore-mentioned problems and likewise guarantees an examination of the patient almost without pain.

It is an object of the invention to provide an endoscope having an everting tube design which shows little push-up tendency, wherein the flexibility of the endoscope is not excessively reduced.

### SUMMARY OF THE INVENTION

The foregoing objects are achieved by an endoscope comprising the features of the independent claim. Further advantageous configurations of the present invention are obtained by the features of the dependent claims.

As a consequence, the basic idea of the invention substantially consists in the fact that the endoscope including an endoscope shaft has an everting tube consisting of a radially outer portion and a radially inner portion, the endoscope shaft being at least partly surrounded by the radially inner portion of the everting tube and longitudinal guiding means engaging with each other are formed at the endoscope shaft and/or the everting tube.

A conception of the invention which is close to this basic idea consists in designing the everting tube with profiles forming undercuts extending in the longitudinal direction of the everting tube (for example in the manner of a dovetail guide). The endoscope shaft is designed to have corresponding profiles at least in sections which at least partially extend in the direction of movement of the everting tube and slidingly engage in the profiles at the everting tube. Both profiles engage in such manner that a tensile force can be exerted on the everting tube in the direction of the endoscope shaft and possibly in the longitudinal direction of the shaft so as to prevent the everting tube from lifting off or detaching from the surface of the endoscope shaft. What is decisive in this type of longitudinal guiding means therefore is to design the undercuts to be not transverse to the direction of movement of the everting tube, as provided in the prior art mentioned in the beginning by arranging a snap ring, but to provide the undercuts in the longitudinal direction of movement of the everting tube, similarly to a travel rail, whereby the entire holding surface by which the everting tube is held at the endoscope shaft is enlarged and, moreover, the sliding friction between the two profiles is reduced in the case of a relative movement between the everting tube and the endoscope shaft.

A further conception of the invention in accordance with the aforementioned basic idea consists in the fact that the respective longitudinal guiding means are, on the one hand, in the form of a continuous conveyor extending along the endoscope shaft in the longitudinal direction thereof and, on the other hand, in the form of an engaging means positively or frictionally connected to the continuous conveyor, which engaging means is provided at the everting tube and, more exactly, at one side of the everting tube. In this way, likewise an engagement of the everting tube with the endoscope shaft is brought about in the longitudinal direction of movement of the endoscope shaft, wherein in this case the continuous conveyor represents a self-moving longitudinal guiding means (active) in contrast to the afore-described conception of the invention, namely the arrangement of a rail guide (passive).

### BRIEF DESCRIPTION OF THE DRAWINGS

Hereinafter the invention will be described in detail by way of preferred embodiments with reference to the accompanying drawings, in which

FIG. 1 shows an endoscope comprising an everting tube and an endoscope shaft according to the first embodiment of the present invention,

FIG. 2 shows an endoscope according to the first embodiment of the present invention, in addition an everting tube drive for the endoscope being provided,

FIG. 3 shows an endoscope according to a second embodiment of the present invention, in addition a shaft drive for the endoscope being provided,

FIG. 3a shows an embodiment of the continuous conveyor,

FIG. 4 shows a third embodiment of an endoscope according to the invention,

FIG. 5 shows a perspective view of an everting tube according to the invention in accordance with a fourth preferred embodiment of the invention,

FIG. 6 shows the cross-sectional view of the everting tube of FIG. 5,

FIG. 7 shows a perspective cut-out of a radial projection of an endoscope shaft in accordance with the fourth embodiment of the invention,

FIG. 8 shows a perspective view of a central portion of the endoscope shaft including the mounted everting tube in accordance with the fourth embodiment of the invention,

FIG. 9 shows a perspective view of an everting tube in accordance with a fifth preferred embodiment of the invention,

FIG. 10 shows a cross-sectional view of the everting tube according to the fifth preferred embodiment of the invention, and

FIG. 11 shows a radial projection of the endoscope shaft according to the fifth preferred embodiment.

### DETAILED DESCRIPTION OF THE PREFERRED EMBODIMENTS

In accordance with the invention, an endoscope 1 is provided which in general includes an endoscope shaft 3 and an everting tube 2 and which is used for examination of a canal-shaped cavity. The endoscope shaft 3 having an end leading in the inserting direction, i.e. the distal end and a trailing end, i.e. the proximal end, furthermore includes at least one optical means for optically detecting the cavity to be examined and can further optionally include a lighting means, a means for treatment, a spraying means etc. (all of which are not shown in the Figures). The everting tube 3 consists, as is known already from prior art, of a radially inner portion 4 which is extended along at least one distal everting portion to form a radially outer portion 5.

In order to satisfy the demands of high flexibility, both for the endoscope shaft and for the everting tube preferably highly flexible materials are used, such as for instance EPTFE, whereby the everting tube 2 and the endoscope shaft 3 can easily adapt to the course of the canal to be examined, for instance the intestines of a patient. The endoscope shaft 3 is directly surrounded at least partly by the radially inner portion 4 of the everting tube, wherein longitudinal guiding means extending in the longitudinal direction of the endoscope shaft and engaging with each other are formed at the endoscope shaft 3 and the everting tube 2.

During insertion into the hollow canal, for instance the intestines, the radially outer portion 5 of the everting tube 2 is adjacent to the wall of the hollow canal, for instance the intestinal wall, and thus is continuously in a rest position with respect to the endoscope shaft 3. The radially inner portion 4 of the everting tube 2 is in contact with the endoscope shaft 3. Furthermore, the radially inner portion 4 is the one that moves relative to the radially outer portion 5 at a predetermined speed. By everting the tube 2 the radially inner portion 4 is moved along during movement in the direction of the course of the hollow canal and simultaneous rest position of the radially outer portion 5 by a quasi “unrolling motion” and thus is changed into the radially outer portion.

In accordance with the first embodiment of the invention, between the radially inner portion 4 of the everting tube 2 and the endoscope shaft 3 there is an operative connection which is composed, on the one hand, of an engaging means 6 and, on the other hand, of a continuous conveyor 7 disposed at the endoscope shaft 3 which extends in the longitudinal direction of the endoscope shaft and acts as a longitudinal guiding means. The engaging means 6 consists of two meshing components disposed at the everting tube 3 and at the continuous conveyor 7, respectively. The component of the engaging means 6 disposed at the everting tube is arranged merely at one side of the everting tube, i.e. at the outside of the everting tube and extends at least in sections along the radially inner tube portion and also the radially outer tube portion, if necessary.

The engaging means 6 may have different embodiments, for instance a positive or a frictional embodiment or else a combined form of the two foregoing embodiments, whereby the engagement is produced by the correspondingly designed continuous conveyor. For instance, the engaging means may be a mixed form such as a Velcro connection, wherein at the outside of the everting tube a longitudinally extending Velcro strap is arranged, while the continuous conveyor in this case is-equipped with the corresponding counter-component of the Velcro strap. It is also imaginable, however, to design the engaging means of projections and step-backs which are arranged optionally or alternately in the everting tube and at the continuous conveyor and engage in a positive as well as flexible and thus detachable manner.

The simplest variant of such engaging means would be the electrostatic charging of the outside of the everting tube and/or of the continuous conveyor so as to generate an attractive force on the respective other part.

The continuous conveyor itself is disposed, as mentioned in the foregoing, at the endoscope shaft 3 and includes a radially inner portion and a radially outer portion. In this context, several arrangements of the continuous conveyor with respect to the everting tube are possible. The arrangement described in this embodiment describes a continuous conveyor which engages in the engaging means component disposed at the everting tube merely at a portion, namely the radially outer portion of the continuous conveyor. I.e. the continuous conveyor is provided at least with a portion between the endoscope shaft 3 and the everting tube 2, whereas in the fourth embodiment described hereinafter merely a portion of the continuous conveyor is disposed between the endoscope shaft 3 and the everting tube 2 and a further radially outer portion of the continuous conveyor is disposed at the radially outer side of the radially outer portion of the everting tube, i.e. the continuous conveyor surrounds the everting tube.

Just as in the case of the engaging means, various embodiments are possible for the continuous conveyor. It may be formed by an endless conveying means, for instance a belt, a strap, preferably a spherical strap, a filament or a chain etc. But likewise a continuous conveyor of a different design could be applied in this invention, as long as the engagement with the engaging means component disposed at the everting tube can be brought about in a substantially continuous manner.

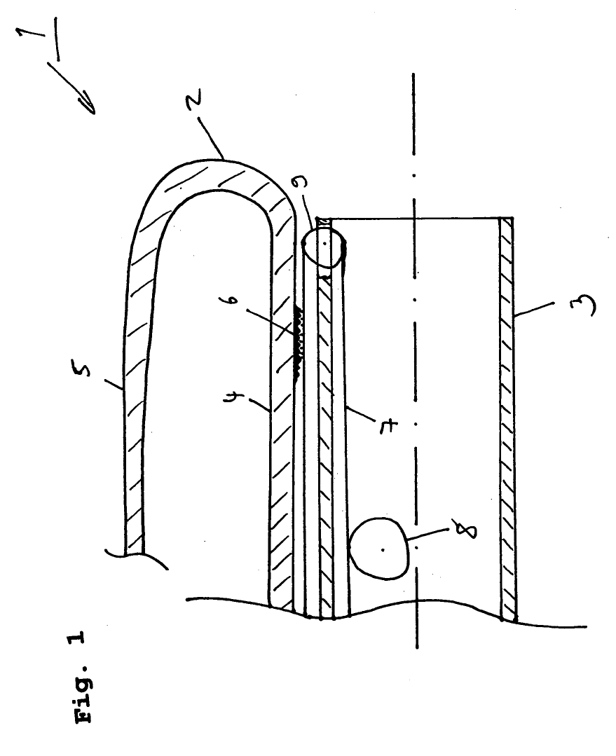

Concerning the afore-mentioned arrangement of the continuous conveyor of this embodiment, the guiding of the continuous conveyor, which is in the form of a strap in this embodiment, can be brought about, for instance, by rollers 9 disposed at both end portions of the endoscope shaft 3 which are spaced apart from each other in the longitudinal direction of the endoscope shaft, as is evident in the FIGS. 1 to 3. In case that the afore-mentioned rollers 9 are disposed at the two end portions of the endoscope shaft 3, one of which corresponds to the respective distal end of the endoscope shaft and the other corresponds to the respective proximal end of the endoscope shaft, the engagement between the continuous conveyor and the component of the engaging means 6 at the everting tube side is brought about or released depending on the direction of movement of the endoscope shaft. In this case, the engaging means component of the continuous conveyor 7 appropriately adapted to the engaging means component of the everting tube 2 is arranged at the outside of the continuous conveyor or, rather, in this embodiment of the conveyor belt. The rollers 9 guiding the continuous conveyor 7 can be arranged at the endoscope shaft 3 in such manner that the continuous conveyor 7 is completely provided radially outside of the endoscope shaft 3 or a respective portion, i.e. the radially outer portion, of the continuous conveyor 7 extends radially outside the endoscope shaft 3 along the longitudinal direction thereof, while the other portion, i.e. the radially inner portion, of the continuous conveyor 7 extends radially inside the endoscope shaft 3, for instance inside a passage formed in the endoscope shaft, as the latter case is schematically shown in FIG. 1.

Likewise a guiding of the continuous conveyor 7 is possible which makes no use of rollers. So a groove can be formed at the outer circumference of the endoscope shaft 3 which is formed along the endoscope shaft in the longitudinal direction thereof and further extends in a passage formed radially inside the endoscope shaft so that a continuous groove is formed. The continuous conveyor can be inserted or embedded in said groove, whereby the guiding function of the continuous conveyor can be assumed solely by the above-described groove instead of the rollers.

By virtue of the continuous conveyor 7 arranged at the endoscope shaft 3 which is guided by the rollers 9 or is disposed in the circumferential groove, as mentioned before, the endoscope shaft 3 can be moved independently of the motion of the everting tube 2, i.e. it can perform a movement relative to the everting tube 2.

The above-described arrangement of the continuous conveyor, due to which the endoscope shaft 3 is movable relative to the everting tube 2, can be driven in different ways to move the everting tube as well as of the endoscope shaft forward. This can be effected, on the one hand, by a drive acting directly upon the endoscope shaft and, on the other hand, by an everting tube drive in the case of which the continuous conveyor is driven quasi via the everting tube.

In accordance with the first embodiment of the present invention, the above-described endoscope is described to be equipped with an everting tube drive directly acting upon the everting tube.

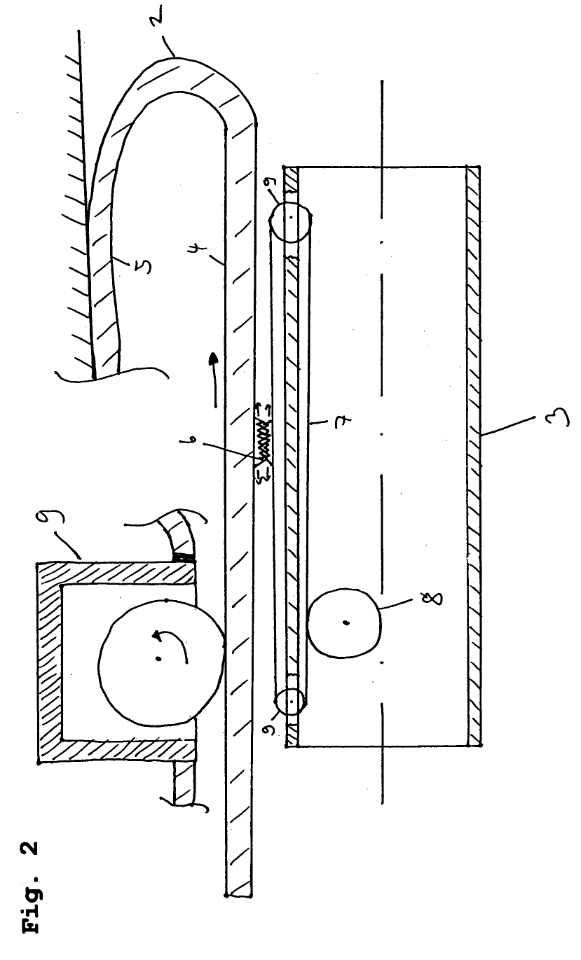

In the present case, the driving of the radially inner portion 4 of the everting tube 2 by a drive means 9 is understood by an everting tube drive, as one can take from FIG. 2. Since, however, according to the invention also a movement of the endoscope shaft relative to the everting tube must be possible, further configurations of the endoscope are necessary.

By driving the radially inner portion 4 of the everting tube 2 in the direction of insertion (corresponding to the course of the hollow canal, for instance the intestines) while the radially outer portion 5 of the everting tube 2 which is adjacent to the wall of the hollow canal, for instance the intestinal wall, at the same time is in a rest position, the everting tube 2 moves forward in a quasi “unrolling” manner. In this way, the everting tube 2 adapts to the canal to be passed, for instance the intestines, without bulging the same during the forward movement. The continuous conveyor 7, in this case the rotating endless strap, is driven forward by means of the engaged state between the continuous conveyor 7 disposed at the endoscope shaft 3 and the radially inner portion 4 of the everting tube 2 without a forward drive being transmitted to the endoscope shaft 3. The strap is running free of any load so-to-speak. In order to obtain a transmission of force to the endoscope shaft 3, for this purpose a type of balancing means 8 (or a synchronizing means) is arranged at the endoscope shaft 3 which means is engaged with the continuous conveyor 7 either via a coupling with one of the rollers 9 or through contact with the endless strap.

Consequently, the balancing means 8 consists of a gear mechanism or a balancing motor, whereby the forward speed of the endless strap is reduced such that the forward speed of the endoscope shaft adapts to the front everting portion of the everting tube. The use of a balancing motor therefore is moreover advantageous vis-a-vis a mere transmission in so far as hereby the movement of the continuous conveyor 7 can be controlled (to be leading/lagging) relative to the movement of the everting tube.

A blocking of the continuous conveyor 7 by appropriately stopping the balancing means 8 has the effect that the endoscope shaft 3 moves along upon movement of the everting tube 2, however more quickly than the everting tube 2 and the front everting portion thereof, respectively, can move forward in the canal to be passed. Therefore it is necessary to compensate the differential speed between the everting tube 2 and the endoscope shaft 3 by the balancing means 8 so that the endoscope shaft 3 is moved forward at the same (or a lower) speed as (than) the everting tube 2 progresses.

Therefore, the balancing means 8 applies a relative movement to the continuous conveyor 7 so that the endoscope shaft 3 would move counter to the inserting direction in the case of standstill of the radially inner portion 4 of the everting tube 2. Since, however, the everting tube 2 moves forward and a relative movement of the endoscope shaft 3 vis-a-vis the everting tube 2 can be produced by the balancing means 8, the position and the speed, respectively, of the endoscope shaft 3 can be controlled. Thus, the endoscope shaft 3 can be prevented from leading by means of an appropriate control of the balancing means 8, so that the everting tube 2 continuously leads ahead of the endoscope shaft 3 or the everting tube and the endoscope shaft move forward at the same speed.

Even when the everting tube 2 stands still, the position of the endoscope shaft 3 can be changed in any way by the balancing means 8. Furthermore the stops used and also required in prior art become superfluous because of such a structure of the endoscope, whereby no decrease in power occurs due to the friction between such stops and the everting tube.

In the present case, one continuous conveyor arranged at the endoscope shaft or an engaging means formed at the everting tube has been described. But two, three or more continuous conveyors can be arranged along the circumference at the endoscope shaft spaced apart at an equal circumferential distance from one another. Accordingly, two, three or more engaging means can be arranged at the inner circumference of the everting tube spaced apart at an equal circumferential distance from one another.

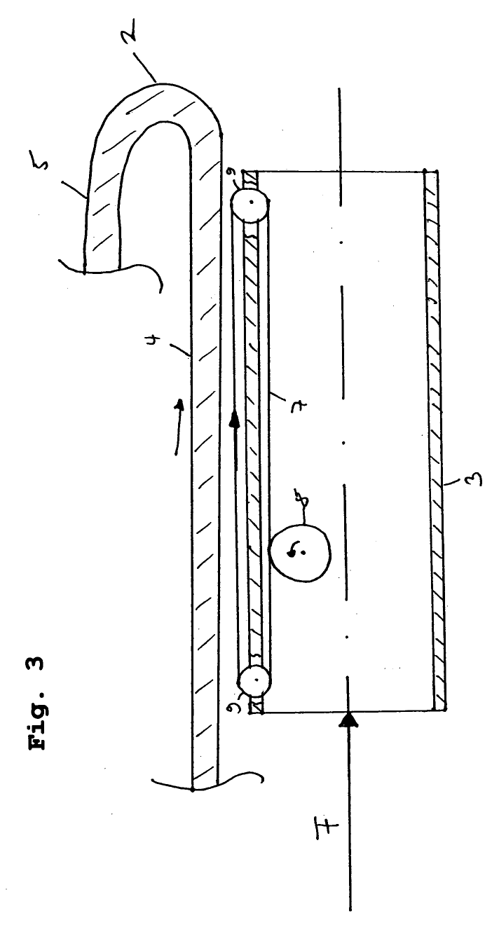

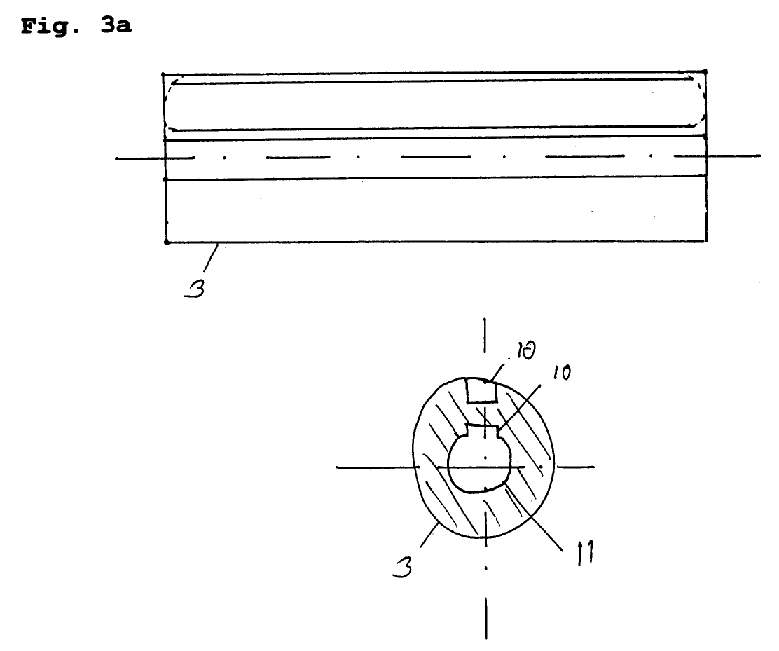

In accordance with a second embodiment of the present invention, the endoscope of the first embodiment is alternatively described to be equipped with a drive acting directly upon the endoscope shaft, as evident from FIG. 3.

Usually it is understood by this type of drive that merely the endoscope shaft is directly driven by a drive means. When applied to the now following second embodiment of the present invention, the endoscope shaft 3 shows a drive means (not shown in detail) which moves the same in the everting tube 2. Via the continuous conveyor 7 engaged with the everting tube through the engaging means 6 the movement of the endoscope shaft 3 is transmitted to the radially inner portion 4 of the everting tube 2 which thereby moves forward, as described in the foregoing, in the canal, for instance the intestines. As the endoscope shaft 3 moves forward more quickly than the everting tube 2, as described before, the endoscope shaft 3 is likewise equipped with the balancing means 8 which moves (drives) the continuous conveyor 7 in such manner that the everting tube 2 performs its quasi “unrolling movement” more quickly, i.e. moves more quickly in the direction of insertion of the endoscope shaft. In this way the endoscope shaft 3 is prevented from leading ahead of the everting tube 2.

The difference of this second embodiment from the foregoing first embodiment of the invention consequently consists in the fact that in this case the speed of the endoscope shaft is preset or rather the forward/inserting movement of the endoscope shaft and, by way of the balancing means 8, the speed of the everting tube are adjusted, whereas in the foregoing embodiment the speed of the everting tube 2 is preset and, by way of the balancing means 8, the speed of the endoscope shaft is adjusted.

In accordance with a third embodiment of the present invention, the continuous conveyor 7, as above-mentioned in the first embodiment of the present invention, is basically arranged and designed in a way directly surrounding the everting tube.

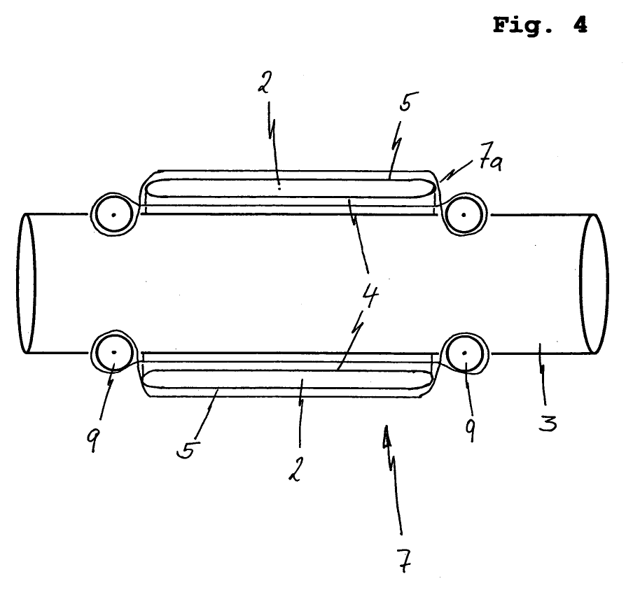

Concretely spoken, the endoscope shaft 3 according to the third embodiment of the invention includes at least one rotating endless strap or filament 7a which is guided by proximal and distal rollers 9 arranged at the endoscope shaft 3 at a distance in the longitudinal direction of the endoscope. As one can take from FIG. 4 in this context, the rollers 9 are supported, similarly to the above-described first and second embodiments of the invention, on the endoscope shaft 3 such that they project slightly from the surface area of the shaft 3.

In contrast to the first and second embodiments, the endless strap 7a according to the third embodiment of the invention does not extend exclusively between the endoscope shaft 3 and the radially inner portion 4 of the everting tube 2, but the part of the endless strap of filament 7a provided outside the endoscope shaft 3 encloses the entire everting tube 2 and thus is actively engaged with the radially outer portion 5 of the everting tube 2.

Concretely spoken, the endless strap or filament 7a according to FIG. 4 is guided at the two longitudinally spaced rollers 9 supported on the endoscope shaft 3. Between the two rollers 9 the double everting tube 2 is arranged. The latter includes at least one longitudinal groove (not shown in detail) which extends at the outside of the everting tube 2 and is completely circumferential in the longitudinal direction of the everting tube 2. The groove forms a longitudinal undercut, for instance by a T-shaped design of the groove in cross-section, and serves for receiving the endless strap or filament 7a. In FIG. 4 the filament 7a is shown at a distance from the everting tube 2. But this is only a schematic diagram. In reality the filament 7a is located inside the groove formed in the everting tube 2 and thus is in close contact with the everting tube. By virtue of the flexibility of the tube material the filament 7a can exit the grooves at both everting portions of the tube 2 and can be guided around the rollers 9.

In this design the everting tube automatically cannot lead via the proximal or distal rollers 9 of the continuous conveyor, because the proximal or distal everting portion thereof is withheld by the respective outer portion of the endless strap or filament 7a. The continuous conveyor 7 in accordance with the third embodiment of the invention accordingly also assumes the function of a front stop and a rear stop, as they are known from the prior art described in the beginning. Moreover, the groove guide of the strap or filament 7a has the effect that the latter does not lift off the endoscope tube when bending the endoscope shaft.

The functioning as regards the drive of the endoscope shaft 3 and the everting tube 2 is equal to that of the first and second embodiments so that reference can be made to the foregoing description passages in this context.

Finally it is pointed out that the endless strap or rotating endless filament is preferably guided around the proximal and distal rollers 9 such that they completely wrap the rollers at least once, as shown in FIG. 4. This type of winding has the advantage that the proximal and distal everting portions of the everting tube 2 are pulled in the direction of the surface area of the endoscope shaft 3 so that the everting tube 2 can be prevented from spreading especially in the distal area. Furthermore, it is advantageous to provide the everting tube 2 with the longitudinal grooves for receiving the endless strap or filament according to the foregoing description so that a better power transmission takes place between the everting tube and the continuous conveyor 7 and moreover the danger of injury by protruding parts especially at the outer portion 5 of the everting tube 2 is reduced.

As an alternative to the represented guiding of the filament or strap 7a around the rollers 9, of course also other possibilities of engagement between the filament and the roller are imaginable. The rollers could show, for example, a type of clamping groove in the circumferential direction in which the filament is inserted.

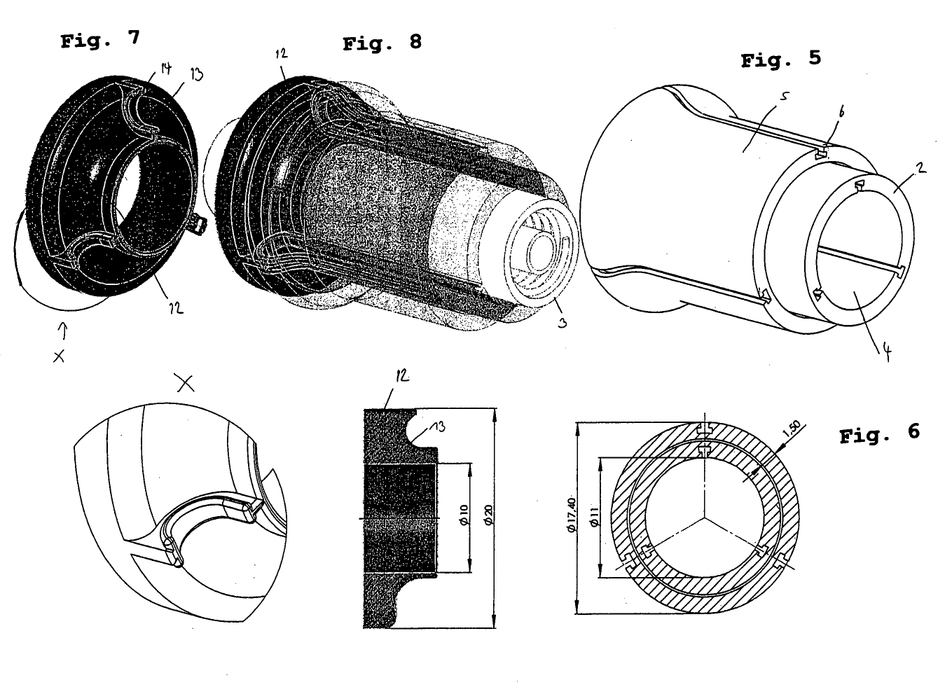

Finally, hereinafter a fourth embodiment of the invention is described by way of the FIGS. 5 to 8.

According to the FIGS. 5 to 8 the endoscope of the fourth embodiment of the invention substantially comprises an endoscope shaft 3 as well as an everting tube 2 at least partly surrounding the same. The everting tube 2 includes a radially inner everting tube portion which is reversed at least at a distal end of the endoscope shaft 3 to form a radially outer everting tube portion. At an outer tube side the everting tube 2 has at least one longitudinal guiding. The latter consists of a slit or a groove extending in the longitudinal direction of the everting tube 2 and forming in its groove bottom a T-undercut in cross-section. As an alternative to that, the longitudinal groove could also be in the form of a dovetail guide or a similar undercut.

In the present case, three longitudinal grooves arranged at an equal circumferential distance from one another are formed. Alternatively to that, also only one, two or more than the three shown grooves could be provided.

As one can take especially from FIG. 5, the grooves are disposed at the side of the everting tube which, after passing the distal everting portion or the radially inner portion, is then located on the outside of the outer everting tube portion or of the radially outer portion.

The endoscope shaft 3 schematically represented in FIG. 8 comprises a coating preferably laminated by means of a helical spring or a spun yarn in which a working passage is formed. At a distal end portion of the endoscope shaft 3 a radial projection 12 is formed which forms a sliding surface 13 facing the everting tube and being preferably adapted to the distal everting portion of the everting tube. In this sliding surface 13 a number of slide rails 14 is formed the cross-section of which corresponds to the cross-section of the grooves formed in the everting tube 2. Moreover the slide rails 14 are spaced apart in the circumferential direction corresponding to the grooves in the everting tube 2.

When mounting the everting tube 2, the latter is pulled onto the endoscope shaft 3 in an initially non-reversed state in such manner that the slide rails 14 provided at the distal end portion of the endoscope shaft 3 which extend in the longitudinal direction of movement of the everting tube 2 and are adapted substantially U-shaped in accordance with the sliding surface 13 and the shape of the distal everting portion of the everting tube, respectively, are threaded into the corresponding grooves. Thus, the everting tube is necessarily reversed at the distal end portion of the endoscope shaft, whereby the outer everting portion is formed which returns in the direction of the proximal end (not shown) of the endoscope shaft 3.

The radial projection 12 arranged at the distal end portion of the endoscope shaft 3 and the appropriately designed slide rails 14 formed at the projection, respectively, consequently are slidingly engaged with the grooves formed in the everting tube 2, wherein the undercuts at the groove bottom formed by the T-shape are brought into guiding engagement with the corresponding T-shaped projections of the slide rails 14 and thus prevent the slide rails 14 from being pulled out of the groove of the everting tube 2. In this way, a tensile or stretch force acting on the everting tube 2 in the longitudinal direction of movement can be exerted by the radial projection of the endoscope shaft 3.

In the FIGS. 9 to 11 a fifth embodiment is represented as an alternative to the fourth embodiment. The design principle of the fifth embodiment substantially corresponds to that of the above-described fourth embodiment, wherein the slide rail arrangement and the groove arrangement, respectively, at the everting tube and at the distally disposed projection of the endoscope shaft, respectively, are exchanged. In other words, according to the sixth embodiment of the invention, the everting tube 2 includes longitudinally extending, preferably T-shaped (or differently designed, such as dovetail, L-shaped etc.) slide rails 17 which are radially projecting at one side of the surface area.

In this case, too, the side on which the slide rails 17 are arranged at the everting tube 2 is chosen such that they project outwardly in the front everting portion as well as from the outer everting portion of the everting tube 2.

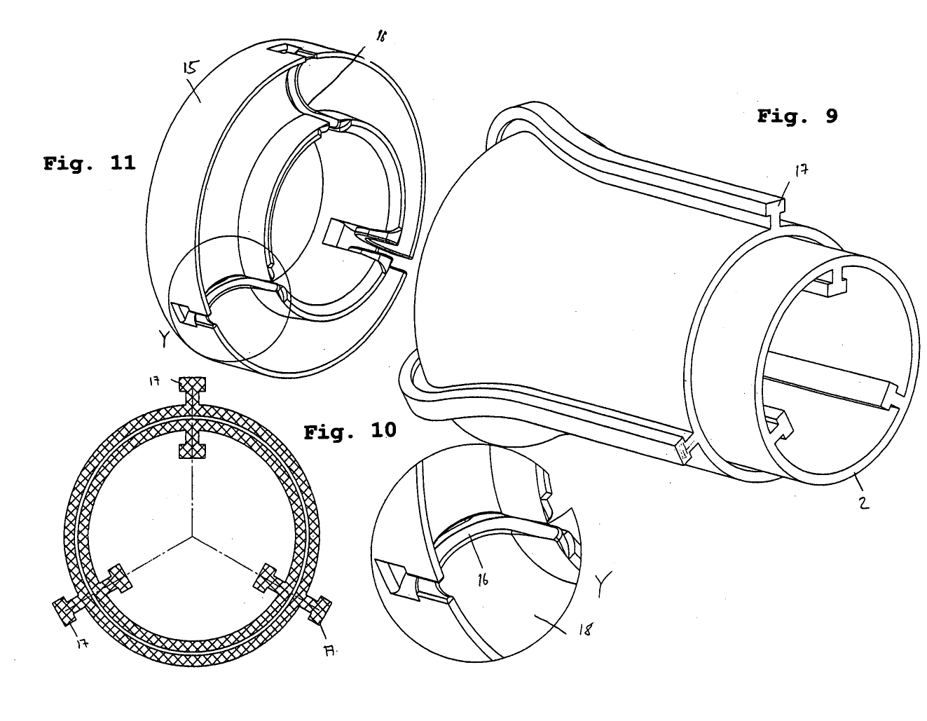

In FIG. 9 the slide rails 17 provided at the everting tube are T-shaped in cross-section and, as shown in FIG. 10, are evenly spaced apart from one another in the circumferential direction. Preferably three slide rails 17 are provided, wherein the number can also be varied to two or more than three slide rails 17. As an alternative to the T-shape, each slide rail may also be dovetail-shaped or have a different shape, as long as an undercut suited for guiding the everting tube is formed.

Moreover, each slide rail 17 is continuously represented in the enclosed FIG. 9 in the longitudinal direction of the everting tube. It is referred to the fact, however, that for obtaining an as narrow everting portion as possible and moreover a high flexibility of the entire endoscope the slide rails 17 can also be interrupted to form a plurality of slide rail pieces, whereby desired bends are defined to reduce the bending stiffness of the everting tube.

FIG. 11 shows the radial projection 15 as it is formed and arranged at the distal end portion of the endoscope shaft 3. Also with this projection 15 a sliding surface 18 facing the everting tube 2 is formed which comes substantially close to the shape of the front everting portion and therefore adopts a substantially trough-shaped contour. This contour corresponds, by the way, also to that of the fifth embodiment.

Contrary to the fourth embodiment, however, on this sliding surface 18 grooves evenly spaced apart in the circumferential direction are formed which extend in the sliding direction of the everting tube 2, i.e. traversing the trough-shaped sliding surface, and whose groove bottom is provided with undercuts in T-shape or formed corresponding to the slide rails 17. Consequently, the shape and the distances of the grooves provided in the radial projection are such that when the everting tube 2 is pulled onto the endoscope shaft 3 the slide rails 17 provided thereon can be threaded into the grooves 16 of the radial projection, whereby the everting tube 2 is automatically reversed into the outer everting tube portion in the area of the radial projection 15. Moreover, in conformity with the above-described fifth embodiment, a tensile force which extends the everting tube 2 in the longitudinal direction of the endoscope shaft 3 can be exerted on the everting tube via the radial projection 15 and the grooves formed therein.

An endoscope of this type can be operated as follows:

Without everting tube drive (passive system): An endoscope having the described basic structure according to the fourth and fifth embodiments can be manually inserted in a canal-shaped cavity such as, for instance, the intestines of a patient. In this case, a feed force is manually applied directly to the endoscope shaft at the proximal end portion thereof, thereby the endoscope shaft 3 being driven forward into the intestines of the patient. The high flexibility of the endoscope shaft as well as the arrangement of a further device such as, for instance, a deflecting disposed at the distal end of the endoscope shaft, permit to guide the endoscope shaft 3 also around bends of the intestinal system without the endoscope shaft 3 causing a bulging of the intestinal loops.

In the case of such propulsion the everting tube is driven in a quasi passive manner by the fact that the outer everting portion thereof so-to-speak adheres to the intestinal wall and thus pulls the inner everting tube portion in the direction of the distal radial projection of the endoscope shaft at a speed which is double with respect to the speed of the endoscope shaft. This tensile force is transmitted via the radial projection and the guide rails or grooves formed thereon, respectively, according to the afore-described fifth and sixth embodiment to the distal everting portion and the grooves and/or slide rails of the everting tube formed thereon, wherein the everting tube glides off along the sliding surface of the radial projection. By virtue of the longitudinal guiding at the everting tube 2 and at the radial projection of the endoscope shaft 3, the sliding friction automatically occurring when the tensile forces are transmitted to the front everting portion of the everting tube is very low so that the total feed force to be applied to the endoscope shaft for propelling the same into the intestines is correspondingly low.

With an additional everting tube drive (active system):

As an alternative to the afore-described driving mode of the endoscope, it is also possible, of course, to equip the endoscope according to the invention with a drive mechanism for motor propulsion. In this case a motor drive force would be applied to the everting tube 2 which moves the same in the longitudinal direction of the endoscope shaft 3. This drive mechanism is designed such that at least part of the drive force is preferably applied to the endoscope shaft 3 itself which causes a forward movement of the endoscope shaft 3. For obtaining this drive motion, for instance two drives could be provided which are adapted to each other as to their speed and each of which drives the endoscope shaft 3 and the endoscope tube (everting tube 2), respectively. Drive means of this type are known from prior art, for instance according to the publications cited in the beginning so that reference can be made to the corresponding documents in this context.

In the case of such active system, i.e. an endoscope in which the everting tube 2 is actively driven by a drive means, part of the drive force applied to the everting tube 2 is transmitted via the distal radial projection indirectly to the endoscope shaft 3 for the propulsion thereof. In addition, the endoscope shaft 3 itself is directly moved forward by the drive mechanism, whereby the endoscope shaft can possibly lead ahead of the distal everting portion of the everting tube. By forming the longitudinal guiding of the above-described groove and guide rail combination at the everting tube as well as at least at the distal radial projection the everting tube 2 and the distal everting portion thereof, respectively, are quasi pulled along with the endoscope shaft so that the everting tube is effectively prevented from folding or dragging. Since moreover the slip friction can be kept low by designing the guide means according to the invention as a longitudinal guiding, the drive forces for moving the endoscope forward can be adjusted to be appropriately small so that the endoscope shaft which preferably consists of an EPTFE material can be largely prevented from buckling.

Furthermore, the groove and the slide rails can be provided with a laminated reinforcement so as to avoid an undesired release of the engaged state between the respective longitudinal guide means. The reinforcement may be of any type as long as the engaged state between rail/groove is reinforced thereby. Preferably a wire, preferably a metal wire, incorporated in the rail and/or in the groove is used. Thus, when force acting in the direction of releasing the engagement occurs, the maintenance of the engaged state is safeguarded.

It is finally referred to the fact that the above-described longitudinal guiding consisting of a guide rail and a corresponding groove including an undercut by no means needs to be provided only in the area of the distal radially extending projection, but can also be formed instead or in addition thereto along the endoscope shaft at the outer surface area thereof. That is to say that appropriately designed guide rails or grooves are formed at the outer surface area of the endoscope shaft to form the afore-described grooves or guide rails at the distal radial projection in which the endoscope tube is threaded or rather inserted in the area of its inner tube portion. This type of longitudinal guiding would be possible also without the arrangement of corresponding guide rails or grooves at the distal projection of the endoscope shaft, wherein, however, increased frictional forces have to be expected at the distal outlet area of the everting tube, i.e. in the area in which the guide rails or grooves at the distal end portion of the endoscope shaft leave the corresponding counterparts. In order to avoid this in an efficient manner, also a combination of an endoscope shaft of this design including a radial projection according to the afore-described third or fourth embodiment is possible.

1. An endoscope comprising an everting tube including a radially outer portion and a radially inner portion and an endoscope shaft arranged at least partly in the everting tube, wherein longitudinal guiding means which engage with each other are formed at each of the endoscope shaft or a component fixed thereto and the everting tube.

2. An endoscope according to claim 1, wherein one of the longitudinal guiding means forms an undercut which is in sliding engagement with the respectively shaped other longitudinal guiding means.

3. An endoscope according to claim 1, wherein the longitudinal guiding means formed at the endoscope shaft is a continuous conveyor, whereas the longitudinal guiding means formed at the everting tube is an engaging means.

4. An endoscope according to claim 3, wherein a drive means propels the radially inner portion in a longitudinal direction while the radially outer portion at the same time is in a rest position and a balancing means arranged at the endoscope shaft drives the continuous conveyor engaged with the engaging means, whereby a movement of the endoscope shaft relative to the everting tube is controllable.

5. An endoscope according to claim 3, wherein a drive means moves the endoscope shaft in a longitudinal direction and a balancing means arranged at the endoscope shaft drives the continuous conveyor engaged with the engaging means, whereby a movement of the radially inner portion relative to the endoscope shaft is controllable while the radially outer portion at the same time is in a rest position.

6. An endoscope according to claim 3, wherein the continuous conveyor surrounds the everting tube and is preferably guided by rollers arranged at two ends of the endoscope shaft which are wrapped, at least partly, preferably once or several times by the continuous conveyor.

7. An endoscope according to claim 6, wherein the continuous conveyor is further guided by a groove extending at an outer circumference of the endoscope shaft in a longitudinal direction thereof.

8. An endoscope according to claim 6, wherein a drive means either drives the continuous conveyor or moves the endoscope shaft forward in a longitudinal direction thereof.

9. An endoscope according to claim 4, wherein the continuous conveyor is guided by deflection rollers disposed at the endoscope shaft, wherein a radially outer portion of the continuous conveyor is engaged with the engaging means disposed at the radially inner portion of the everting tube and a radially inner portion of the continuous conveyor is engaged with the balancing means disposed at the endoscope shaft.

10. An endoscope according to claim 5, wherein the continuous conveyor is guided by deflection rollers disposed at the endoscope shaft, wherein a radially outer portion of the continuous conveyor is engaged with the engaging means disposed at the radially inner portion of the everting tube and a radially inner portion of the continuous conveyor is engaged with the balancing means disposed at the endoscope shaft.

11. An endoscope according to claim 4, wherein the endoscope shaft includes a groove extending in the longitudinal direction thereof at the outer circumference which further extends in a passage formed in the endoscope shaft in the longitudinal direction thereof so that a continuous closed groove is formed into which the continuous conveyor is inserted.

12. An endoscope according to claim 5, wherein the endoscope shaft includes a groove extending in the longitudinal direction thereof at the outer circumference which further extends in a passage formed in the endoscope shaft in the longitudinal direction thereof so that a continuous closed groove is formed into which the continuous conveyor is inserted.

13. An endoscope according to claim 3, wherein the continuous conveyor can be in the form of a belt, strap, preferably a spherical strap, filament or chain.

14. An endoscope according to claim 2, wherein the sliding engagement between the longitudinal guiding means is brought about by forming a recess constituting an undercut, preferably a groove and by forming an appropriately shaped projection, preferably a rail, inserted in the recess.

15. An endoscope according to claim 2, wherein the undercut is preferably formed by a T-shaped cross-section or a dovetail cross-section.

16. An endoscope according to claim 12, wherein the undercut is preferably formed by a T-shaped cross-section or a dovetail cross-section.

17. An endoscope according to claim 1, wherein the longitudinal guiding means disposed at the everting tube and the longitudinal guiding means disposed at the endoscope shaft have plural, preferably three longitudinal guiding portions which are circumferentially, preferably evenly, spaced apart from one another and arranged such that the respective longitudinal guiding portions of both longitudinal guiding means can be engaged with each other.

18. An endoscope according to claim 2, wherein the longitudinal guiding means disposed at the everting tube and the longitudinal guiding means disposed at the endoscope shaft have plural, preferably three longitudinal guiding portions which are circumferentially, preferably evenly, spaced apart from one another and arranged such that the respective longitudinal guiding portions of both longitudinal guiding means can be engaged with each other.

19. An endoscope according to claim 14, wherein the projection inserted in the recess is formed partially or continuously in the longitudinal direction of the endoscope shaft or the everting tube.

20. An endoscope according to claim 14, wherein a reinforcing means, preferably reinforcing metal clips, are incorporated in the projection and/or in the recess.

21. An endoscope according to claim 2, wherein at an end of the endoscope shaft leading in the direction of movement of the endoscope shaft a radial projection is formed which transforms the radially inner portion of the everting tube by means of a guiding slide surface into the radially outer portion, wherein the guiding slide surface is preferably provided with a guiding means extending the longitudinal guiding means of the endoscope shaft.

22. An endoscope according to claim 14, wherein at an end of the endoscope shaft leading in the direction of movement of the endoscope shaft a radial projection is formed which transforms the radially inner portion of the everting tube by means of a guiding slide surface into the radially outer portion, wherein the guiding slide surface is preferably provided with a guiding means extending the longitudinal guiding means of the endoscope shaft.

23. An endoscope according to claim 1, wherein the endoscope shaft and/or the everting tube are/is made of a highly flexible material, preferably EPTFE.

24. An endoscope according to claim 1, wherein the endoscope shaft has at least an optical means and optionally furthermore a lighting means and/or a means for treatment and/or a spraying means.

25. An endoscope according to claim 2, wherein the endoscope shaft has at least an optical means and optionally furthermore a lighting means and/or a means for treatment and/or a spraying means.
